(19) United States
(12) Patent Application Publication
(10) Pub. No.: US 2021/0046131 A1
(43) Pub. Date: Feb. 18, 2021

(54) WIDE-SPECTRUM SALMONELLA PHAGE AND APPLICATION THEREOF

(71) Applicant: QINGDAO PHAGEPHARM BIO-TECH CO., LTD, Qingdao (CN)

(72) Inventors: Qiang PAN, Qingdao (CN); Hui Ying REN, Qingdao (CN); Hu Zhi SUN, Qingdao (CN); Guang Qin LIU, Qingdao (CN); Cui WANG, Qingdao (CN)

(21) Appl. No.: 16/967,882

(22) PCT Filed: Jan. 16, 2019
(86) PCT No.: PCT/CN2019/071903

§ 371 Date: Aug. 6, 2020

(30) Foreign Application Priority Data
Feb. 7, 2018 (CN) 201810125673.8

(51) Int. Cl. A61K 35/76; A01N 63/40; A23K 10/18; A23K 50/75; A61P 31/04

(52) U.S. Cl. CPC A61K 35/76; A01N 63/40; A61P 31/04; A23K 50/75; A23K 10/18

(57) ABSTRACT

The present invention relates to a Salmonella bacteriophage, in particular to a Siphoviridae bacteriophage with a wide host spectrum and a strong lytic activity against Salmonella pullorum. The above-mentioned Salmonella pullorum bacteriophage is named SP4, and is deposited at the China General Microbiological Culture Collection Center, the deposit date is Jul. 27, 2017 and the deposit number is CGMCC No, 14332, The bacteriophage has a strong lysis effect on Salmonella, and the bacteriophage can also reduce the mortality rate of chicks infected with Salmonella pullorum, The preparation can be used alone or as a cocktail, and provides a safe, non-toxic and side-effect-free and residual effect-free bacteriophage product for the treatment of the infections caused by Salmonella of chicken origin, Salmonella of duck origin, Salmonella of mink origin, Salmonella of food origin and Salmonella of pig origin.

### TECHNICAL FIELD

The present invention belongs to the field of biotechnology, and relates to a Siphoviridae bacteriophage with a wide host spectrum and a strong lytic activity against Salmonella pullorum and its application in a breeding environment.

### BACKGROUND ART

Pullorum disease is an acute systemic disease caused by Salmonella pullorum. It is one of the most serious bacterial infectious diseases that harm the chicken industry and mainly transmitted vertically through eggs, and can also infect through the digestive tract and respiratory tract. The eggs infected with pullorum disease often show dead embryos or weak embryos, and cannot hatch or die after hatch, and generally have no special clinical symptoms. Chick infection is generally acute, with a peak incidence between 7 and 10 days of age. There are three common clinical symptoms: acute septic symptom, arthritic symptom and neurological symptom. The clinical symptoms are mostly thin and white paste-like stools, the emergence of paste anus phenomenon and the death of sick young mostly due to breathing difficulties and heart failure. 2 to 3 weeks old chicks have a higher infection mortality rate. Chickens over 4 weeks of age infected with pullorum disease generally have fewer deaths. Adult chickens are dominated by local or chronic infections and recessive infections. Hens show decreased egg production and the mortality rate after infection is low, but the bacteria can be carried and discharged for a long time.

At present, the main methods for clinical prevention and treatment of pullorum disease are regular quarantine, purification of breeding flocks, all-in and all-out and self-breeding and self-cultivating management measures and production models. Because the Salmonella disease can spread horizontally and vertically, the above prevention effects are not ideal. At present, most farms focus on drug prevention. The prevention using large amounts of antibiotics leads to a serious problem of bacterial drug resistance. After controlling Salmonella contamination has become a worldwide problem, the problem of drug resistance of Salmonella is also a global problem. Therefore, research and development of new products that are pollution-free, residue-free, safe, and can replace antibiotics to solve the problem of Salmonella contamination have attracted wide attention of all circles.

Bacteriophages are widely distributed in nature, with simple preparation technology, short research and development period, difficult to develop drug resistance and low cost. Bacteriophages have a strong specificity, generally only infect pathogenic bacteria of certain species without destroying normal flora; bacteriophages also have a very strong proliferative capacity and can be used as a therapeutic agent to continuously expand the efficacy. Current studies have not found that bacteriophage treatment will cause serious side effects, nor has there been any reports of allergies to oral bacteriophages. The most important thing is that bacteriophage treatment is not limited by the bacterial drug resistance. Bacteriophages have a completely different bactericidal mechanism from antibiotics and are unaffected by antibiotic resistance that bacteria have acquired. Bacteriophages only act at the site of bacterial infection and decreases with the death of pathogens until they disappear without causing secondary contamination.

### SUMMARY OF THE INVENTION

One object of the present invention is to provide a Siphoviridae bacteriophage of Salmonella pullorum with a wide host spectrum.

Another object of the present invention is to provide an effective product for the prevention and treatment of Salmonella pullorum disease in the current breeding industry. The present invention provides a Siphoviridae bacteriophage with a wide host spectrum and a strong lytic activity against Salmonella pullorum and develops a bacteriophage preparation with a strong lytic activity against Salmonella pullorum, which can be used alone or in cocktails. The present invention provides a safe, non-toxic and side-effect-free and residual effect-free bacteriophage product for the treatment of Salmonella pullorum infection, and provides a source of bacteriophage for the industrial production of bacteriophage preparations.

Another object of the present invention is to provide a pollution-free, residue-free, environmentally-friendly and effective prevention and treatment method. The bacteriophage SP4 is prepared as a liquid or a lyophilized powder, which can be used to kill Salmonella in animals in vitro and in vivo and in breeding environments by oral administration or spraying, alone or in combination with other bacteriophages.

The objects of the present invention are achieved as follows: a Siphoviridae bacteriophage with a wide host spectrum and a strong lytic activity against Salmonella pullorum, whose deposit number is CGMCC No. 14332, depositary institution is the China General Microbiological Culture Collection Center and deposit date is Jul. 27, 2017. The bacteriophage is named SP4, and it has lytic activity against Salmonella of chicken origin, Salmonella of pig origin, Salmonella of duck origin, Salmonella of mink origin and Salmonella of food origin.

The bacteriophage has a polyhedral head structure and a non-constrictive tail. The head diameter is about 53 nm and the tail length is about 108 nm. The bacteriophage can form a large transparent plaque on the double-layer agar medium plate, without a halo ring around it, but with clear and regular edges and a diameter of about 2-3 mm. The bacteriophage belongs to the Siphoviridae family.

In the present invention, the activity of the bacteriophage is stable when placed at 40-50° C. for 60 min, the titer is reduced by about two orders of magnitude when placed at 80° C. for 20 min, and the bacteriophage is inactivated when placed at 70-80° C. for 60 min. The activity of the bacteriophage is stable at pH 6-9.

Use of the bacteriophage is also provided, wherein the purified bacteriophage can lyse Salmonella pullorum, and lyse 23 strains of the collected 24 strains of Salmonella pullorum with a lysis rate of 95.83%.

In the use of the bacteriophage, the purified bacteriophage can lyse Salmonella from different host sources. The bacteriophage SP4 can lyse 64 of the 104 Salmonella strains (28 Salmonella strains isolated from pigs, 11 Salmonella strains isolated from ducks, 14 Salmonella strains isolated from mink, 46 Salmonella strains isolated from chickens, and 5 Salmonella strains isolated from foods). The purified bacteriophage can lyse 6 of the 28 strains isolated from pigs (lysis rate is 21.43%), 41 of the 46 strains isolated from chickens (lysis rate is 89.13%), 9 of the 11 strains isolated from ducks (lysis rate is 81.81%), 7 of the 14 strains isolated from mink (lysis rate is 50%), 1 of the 5 strains isolated from foods (lysis rate is 20%), 23 of the 24 Salmonella pullorum strains of the 46 Salmonella strains isolated from chickens (lysis rate is as high as 95.83%).

In the uses of the bacteriophage, use of the bacteriophage in chicks infected with Salmonella pullorum can reduce the mortality rate by 60% within 2 weeks.

In the uses of the bacteriophage, the bacteriophage can also be used for the control of the infections caused by Salmonella of pig origin, Salmonella of duck origin, Salmonella of mink origin and Salmonella of food origin.

In the uses of the bacteriophage, the purified bacteriophage is prepared in the form of liquid or lyophilized powder, and used for the control of Salmonella infections from different sources by oral administration or spraying, alone or in combination with other bacteriophages or antibiotics.

The present invention has the advantages that the isolated Salmonella pullorum bacteriophage has a lytic activity against Salmonella of chicken origin, Salmonella of pig origin, Salmonella of duck origin, Salmonella of mink origin and Salmonella of food origin, and thus it is a wide host spectrum bacteriophage. The present invention provides a new type of environmentally-friendly products and means for preventing and treating poultry salmonella diseases. The chick safety test proves that the bacteriophage has no toxic or side effects and has high safety. In the morbidity test, the group administered with the bacteriophage has a significantly reduced mortality of chicks.

### DESCRIPTION OF THE DRAWINGS

FIG. 1 is an electron micrograph of SP4 bacteriophage.

FIG. 2 is a picture of SP4 plaques.

FIG. 3 is a picture showing the digestion pattern of SP4 bacteriophage, wherein, Lane M: DL 2000 DNA Marker; Lane 1: SP4 nucleic acid+RNaseA; Lane 2: SP4 nucleic acid+DNase I; Lane 3: SP4 nucleic acid; Lane 4: SP4 nucleic acid+BAL31.

FIG. 4 shows the thermal stability of SP4 bacteriophage.

FIG. 5 shows the pH stability of SP4 bacteriophage.

FIG. 6 is a one-step growth curve of SP4 bacteriophage.

### DETAILED DESCRIPTION OF THE INVENTION

The present invention will be further described with reference to the specific examples below. It should be understood that these examples are only used to illustrate the present invention, not to limit the scope of the present invention. Unless otherwise specified, the experimental methods used in the following examples are conventional methods.

### Example 1: Isolation and Preparation of Bacteriophage SP4

The fecal sewage sample in the present invention was collected from a chicken farm in Shandong Province;

The host strain was Salmonella pullorum CVCC 533.

1. Recovery of Host Strain CVCC 533 and Preparation of its Proliferation Solution

The bacteria solution of the host strain CVCC 533 was picked, and streaked on SS agar medium using the three-zone streak plate cultivation method, and cultured in a 37° C. incubator for 16 to 24 hours to obtain single colonies. Single colonies were picked and inoculated into 5 mL of LB broth contained in test tubes, and incubated at 37° C. with shaking at 170 rpm overnight to obtain a proliferation solution.

2. Treatment of Fecal Sewage Sample

500 μL of the host strain CVCC 533 was added to 50 mL of the fecal sewage from the chicken farm, and then LB medium was added to a final volume of 200 mL. The sample was soaked and incubated overnight at 37° C. The next day, 5 mL of the resulting liquid was taken out, centrifuged at 10,000 rpm for 10 min, and the supernatant was filtered through a 0.22 μm sterile microporous membrane to obtain a bacteriophage stock solution, which was then stored at 4° C.

3. Bacteriophage Isolation

The bacteriophage was isolated by the double-layer plate method and the bacteriophage stock solution was serially diluted 10-fold. 100 μL of the 10⁻² fold and 10⁻⁴ fold dilutions were mixed with 200 μL of the host strain CVCC 533 proliferation solution, respectively. After incubating at 37° C. for 5 min, the solutions were placed on top agar (agar concentration: 0.7%) warmed at about 50° C. After mixing, the resulting mixtures were quickly poured onto bottom agar (agar concentration: 1.5%) in the dish, respectively. The plates were shaken and placed horizontally until the medium was solidified. After culturing at 37° C. for 6-8 h with the plates upside down, two-layer plates with plaque formation were obtained.

### Example 2: Bacteriophage Proliferation and Purification

1. Bacteriophage Proliferation

A single plaque was picked on the two-layer plate with plaque formation with sterilized tweezers and placed in 1 mL of LB broth and incubated in a 40° C. water bath for 30 min to obtain a bacteriophage leaching solution. 200 μL of the bacteriophage leaching solution and 200 μL of the host strain proliferation solution were added to 5 mL of liquid LB medium, and cultured at 37° C. with shaking at 170 rpm until the liquid became clear. The clear liquid was centrifuged at 10,000 rpm for 10 min, and the supernatant was collected and then filtered using a 0.22 μm sterile microporous membrane to obtain a bacteriophage proliferation solution.

2. Bacteriophage Purification

100 μL of the bacteriophage proliferation solution and 200 μL of the host strain CVCC 533 proliferation solution were mixed evenly. After incubating at 37° C. for 5 min, the solution was placed on top agar (agar concentration: 0.7%) warmed at about 50° C. After mixing, the resulting mixture was quickly poured onto bottom agar (agar concentration: 1.5%) in the dish. The plate was shaken and placed horizontally until the medium was solidified. After culturing at 37° C. for 6-8 h with the plate upside down, a two-layer plate with plaque formation was obtained again. A single plaque was picked on the two-layer plate with plaque formation with sterilized tweezers and placed in 1 mL of LB broth and incubated in a 40° C. water bath for 30 min to obtain a bacteriophage leaching solution. 200 of the bacteriophage leaching solution and 200 μL of the host strain proliferation solution were added to 5 mL of liquid LB medium, and cultured at 37° C. with shaking at 170 rpm until the liquid became clear. The clear liquid was centrifuged at 10,000 rpm for 10 min, and the supernatant was collected and then filtered using a 0.22 μm sterile microporous membrane to obtain a bacteriophage proliferation solution. These steps were repeated 3 times to obtain a purified bacteriophage suspension.

### Example 3: Biological Characteristics of Bacteriophage

1. Morphological Characteristics of Bacteriophage SP4

The bacteriophage SP4 was observed under a transmission electron microscope (see FIG. 1). The bacteriophage has a polyhedral head structure and an elongated tail. The head of the bacteriophage has a diameter of about 55 nm, a lateral diameter of about 53 nm, and a tail of about 108 nm. According to the classification of bacteriophages, the morphology of the bacteriophage in the present invention conforms to the characteristics of the Siphoviridae family and the bacteriophage belongs to the Siphoviridae bacteriophage.

2. Culture Characteristics of Bacteriophage SP4

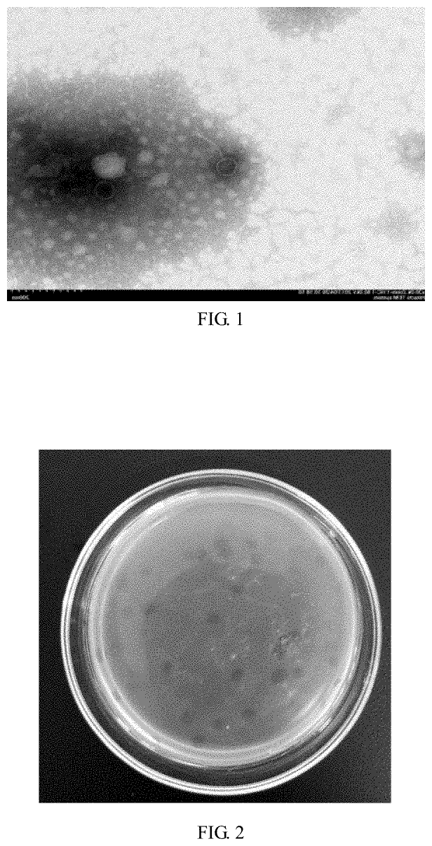

The bacteriophage SP4 can form large translucent plaques on the double-layer agar medium plate, without a halo ring around it, but with clear and regular edges and a diameter of about 2-3 mm (see FIG. 2).

3. Identification of Nucleic Acid Type of Bacteriophage SP4 Genome

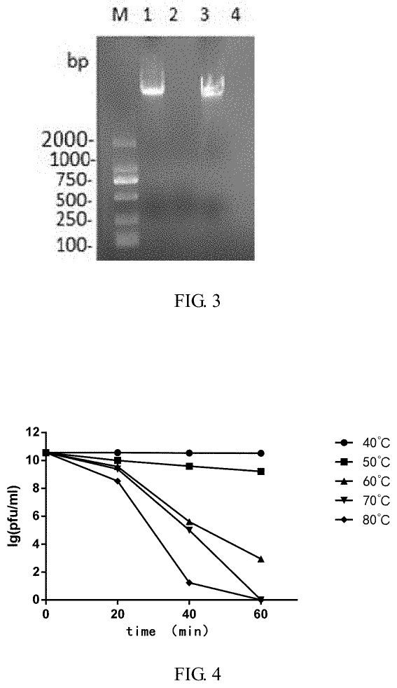

After concentrating the large-scale proliferation of the bacteriophage SP4 by the PEG-NaCl method, the bacteriophage nucleic acid was extracted using the viral genomic DNA/RNA extraction kit, and 5 μL of the SP4 bacteriophage nucleic acid was mixed with 5 μL of DNase I, 5 μL of RNaseA and 5 μL of BAL31 nuclease and 25 μL of BAL31 buffer. The mixture was placed in a 37° C. incubator for 1 h, and the product after the reaction was subjected to 1% agarose gel electrophoresis. According to the analysis of the digestion pattern (see FIG. 3), the bacteriophage SP4 nucleic acid is a double-stranded DNA molecule (dsDNA).

### Example 4: Effect of Temperature and pH on Bacteriophage SP4

100 μL of the bacteriophage SP4 proliferation solution with a titer of 3.8×10¹⁰ PFU/mL was divided into sterile EP tubes, and respectively treated in water baths of 40° C., 50° C., 60° C., 70° C. and 80° C. for 20 min, 40 min and 60 min. Two replicates were set for each temperature. After the treatment, sampling was performed and the samples were immediately placed in an ice bath to cool, and the titer of the bacteriophage was determined by the double-layer plate method after a 10-fold serial dilution. The bacteriophage SP4 thermal stability curve was plotted with temperature as the abscissa and the log value of the bacteriophage titer as the ordinate.

Based on the bacteriophage SP4 of Example 4, 4.5 mL of LB broth with different pH values (2, 3, 4, 5, 6, 7, 8, 9, 10, 11, 12 and 13) was added to a sterile test tube. Then, the above test tubes were placed in a 37° C. water bath, and after the temperature was equilibrated, 500 μL of the bacteriophage proliferation solution was added and mixed. The resulting mixtures were placed in a 37° C. water bath for 1 h, 2 h, and 3 h. The bacteriophage titer was determined by the double-layer plate method for the sample in each tube. Two replicates were set for each pH value. The bacteriophage pH stability curve was plotted with pH value as the abscissa and the log value of the bacteriophage titer as the ordinate.

The thermal stability results (FIG. 4) showed that the bacteriophage SP4 maintained high activity after 60 minutes at 40° C.-50° C., and the titer decreased by 5 titers after 1 hour at 60° C., and the bacteriophage SP4 was completely inactivated after 60 minutes at 70° C.-80° C. It shows that the bacteriophage of the present invention has good thermal stability and can be added to drinking water or feed.

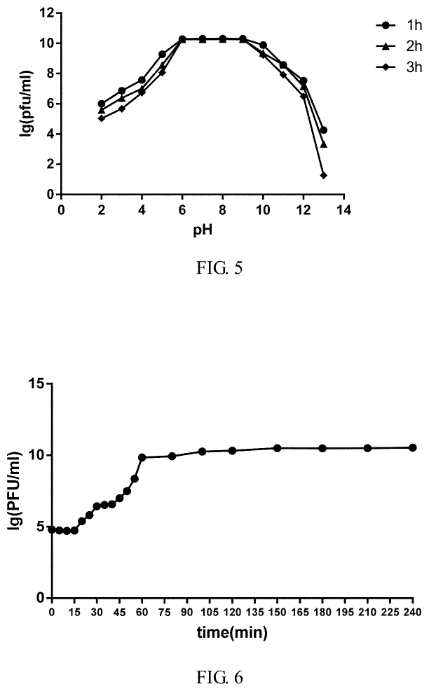

As shown in FIG. 5, the bacteriophage SP4 maintains its original titer after 3 hours in the pH range of 6-9; it is active after 3 hours in the pH ranges of 2-5 and 10-13, and the bacteriophage is more stable in the pH range of 6-9.

### Example 5: One-Step Growth Curve of Bacteriophage SP4

Based on the bacteriophage SP4 of Example 4, 1 mL of the bacteriophage SP4 proliferation solution with a multiplicity of infection of 10 and 1 mL of the fresh proliferation solution of the host strain were mixed well (start timing at this time point), incubated at 37° C. for 5 min, centrifuged at 13000 g for 30 s, the supernatant was removed as much as possible using a micropipette. The precipitate was washed once with 5 mL of LB broth (centrifuged at 13000 g for 30 s), and the supernatant was discarded. The precipitate was suspended with pre-heated LB broth (total volume: 5 mL) and mixed well. The resulting mixture was immediately placed in a 37° C. shaker and cultured at 170 rpm with shaking. From min 0, 150 μL of the culture was taken every 10 min, centrifuged at 10,000 rpm for 1 min, and 100 μL of supernatant was pipetted out and serially diluted 10-fold with normal saline. The bacteriophage titer was measured by the double-layer plate method. Three replicates were set and the results were averaged. The one-step growth curve was plotted with the infection time as the abscissa and the bacteriophage titer in the infection system as the ordinate to obtain the incubation period and burst period of the bacteriophage SP4.

According to the one-step growth curve of the bacteriophage SP4 in FIG. 6, it was found that after the bacteriophage infects the host strain, the titer was basically unchanged within 15 minutes, and the titer was stable at 10⁵ PFU/mL, indicating that the incubation period of the bacteriophage SP4 is about 15 minutes, and the number of bacteriophages increased sharply within 10 to 60 minutes after the bacteriophage infected the host strain; the titer began to stabilize at 60 minutes, and the titer could reach 10¹⁰ PFU/mL at this time point, indicating that the burst period of the bacteriophage SP4 is about 50 minutes and the burst size is 70.

### Example 6: In Vitro Lysis Efficiency of Bacteriophage SP4

The bacteria solution of the host strain stored at −20° C. was picked with a sterile inoculating loop, and streaked on SS agar medium using the three-zone streak plate cultivation method, and cultured in a 37° C. incubator for 16 to 24 hours to obtain single colonies. The recovered single colonies were picked with a sterilized white pipette tip and inoculated into 5 mL of LB broth contained in test tubes, and incubated at 37° C. with shaking at 170 rpm for 16 h to obtain a single host strain suspension. The concentration of the host strain was adjusted to 1×10⁵ CRU/mL, 1 mL of the suspension was mixed with 1 mL of the bacteriophage SP4 at 10⁹, 10⁸, 10⁷, 10⁶, 10⁵ PFU/mL, respectively and placed at room temperature for 30 min. At the same time, a control treatment mixed with 1 μL of SM solution was set. After lightly mixing, the liquid was diluted 10-fold, 100-fold, 1000-fold, and 10000-fold, 100 μL of each gradient diluent was transferred to a common plate, and spread evenly with a sterile spreading rod, and cultured for 16-24 h with the plates upside down. Three replicates were set for each gradient diluent. The number of colonies on each plate was counted.

Bacteriophage lysis efficiency=(1−number of colonies in treatment group/number of colonies in control group)×100%

The in vitro lysis efficiency test of the bacteriophage SP4 found that when the bacteriophage concentration was ≥10⁸ PFU/mL, the host bacteria were completely lysed and when the bacteriophage concentration was ≤10⁷ PFU/mL, the lysis rate gradually decreased.

### Example 7: Analysis of Bacteriophage Lysis Spectrum

Based on the bacteriophage SP4 of Example 4, the single-spot method was used to determine the lysis spectrum of the bacteriophage. The specific steps were as follows: 1 mL of fresh bacteriophage SP4 proliferation solution was centrifuged at 10,000 rpm for 10 min to allow the bacterial debris to settle down; the bacteriophage stock solution was initially selected for testing; 104 strains of Salmonella from different sources kept in the laboratory were respectively picked and subjected to streak cultivation on the SS plates to obtain single colonies; single colonies were picked and inoculated into 5 mL of nutrient broth, incubated at 37° C. and 170 rpm for 12 h to obtain bacteria solution of each strain; 100 μL of each bacteria solution was spread evenly on a common agar plate; after drying, 1 μL of the SP4 bacteriophage proliferation solution was added to the plate dropwise and incubated at 37° C. for 8-12 hours after natural drying. The results were observed.

Through the lysis spectrum determination experiment, it was found that the bacteria solutions selected for the experiment grew well on the plates. The bacteriophage SP4 can lyse 64 of the 104 Salmonella strains (28 Salmonella strains isolated from pigs, 11 Salmonella strains isolated from ducks, 14 Salmonella strains isolated from mink, 46 Salmonella strains isolated from chickens, and 5 Salmonella strains isolated from foods). The bacteriophage can lyse 6 of the 28 strains isolated from pigs (lysis rate was 21.43%), 41 of the 46 strains isolated from chickens (lysis rate was 89.13%), 9 of the 11 strains isolated from ducks (lysis rate was 81.81%), 7 of the 14 strains isolated from mink (lysis rate was 50%), 1 of the 5 strains isolated from foods (lysis rate was 20%), 23 of the 24 Salmonella pullorum strains of the 46 Salmonella strains isolated from chickens (lysis rate was as high as 95.83%). This indicates that the bacteriophage SP4 has a wide lysis spectrum and can be used for the control of Salmonella infections from different sources.

### Example 8: Safety Test of Bacteriophage

Twenty one-day-old SPF chicks were purchased from a chicken farm in Qingdao and randomly divided into two groups: an experimental group and a blank control group. The experimental group was fed with the bacteriophage SP4 proliferation solution at a dose of 1×10¹⁰ PFU/mL/0.25 mL/chick, and the blank control group was fed with an equal volume of sterile saline for 7 consecutive days to observe the behavior and growth of the chicks. After 7 days, 5 chicks were necropsied in each group to observe the changes of viscera, digestive tract and mucosa.

The results showed that the chicks in the experimental group and the control group had the same growth and no adverse reactions. There were no abnormalities in the viscera and digestive tract and mucosa of the two groups in the autopsy, which confirmed that the bacteriophage SP4 was safe and non-toxic.

### Example 9: Chick Treatment Test with Bacteriophage SP4

One hundred and twenty healthy one-day-old chicks were divided into 3 groups, a control group, an infected group and a treatment group, with 40 chicks in each group. The infection was established in the following way: a single colony of Salmonella pullorum CVCC 533 was picked and inoculated into 5 mL of LB medium, cultured for 24 h, and the concentration was adjusted to 1×10⁸ CFU/mL; the control group did not receive the bacteria; each chick in the infected group was given 100 μL of the bacteria orally; in the treatment group, bacteria were given orally at the same time as the bacteriophage SP4; in the infected group, PBS of the same volume was given orally; the experiments were conducted for 5 consecutive days, followed by normal feeding for 14 days. The mortality rate of chicks in each group was observed and calculated.

The results showed that all the chicks in the control group survived, the mortality rate of the chicks in the infected group was 100%, and the mortality rate of the chicks in the treatment group was 40%. It can be seen that the use of the bacteriophage SP4 in chicks infected with Salmonella pullorum can reduce the mortality rate by 60%.

The above examples only illustrate several embodiments of the present invention, and their descriptions are specific and detailed, but they should not be construed as limiting the patent scope of the present invention. It should be noted that, those of ordinary skill in the art, without departing from the concept of the present invention, can also make several modifications and improvements, which all fall within the protection scope of the present invention. Therefore, the protection scope of the present invention shall be defined by the appended claims.

What is claimed is:

1-10. (canceled)

11. A method of treating and/or preventing diseases caused by Salmonella infections comprising: administering the medicament to the subject, wherein the medicament comprises a Salmonella bacteriophage, which has the deposit number of CGMCC No. 14332.

12. The method according to claim 11, wherein the Salmonella is selected from Salmonella of chicken origin, Salmonella of pig origin, Salmonella of duck origin, Salmonella of mink origin and Salmonella of food origin; the Salmonella of chicken origin is selected from Salmonella pullorum, Salmonella gullinarum and Salmonella enteritidis.

13. The method according to claim 11, wherein the diseases caused by Salmonella infections is selected from pullorum disease, avian typhoid, avian paratyphoid, swineparatyphoid, mink salmonellosis, Salmonella typhimurium enteritis.

14. A pharmaceutical composition or feed additive comprising the Salmonella bacteriophage, which has the deposit number of CGMCC No. 14332.

15. The pharmaceutical composition or feed additive according to claim 14, further comprising a pharmaceutically acceptable carrier.

16. The pharmaceutical composition or feed additive according to claim 14, wherein the pharmaceutical preparation form of the pharmaceutical composition is an oral administration dosage form or a spray dosage form or a parenteral administration dosage form.

17. The pharmaceutical composition or feed additive according to claim 14, wherein the pharmaceutical preparation form is oral administration dosage form.

18. The pharmaceutical composition or feed additive according to claim 14, wherein the pharmaceutical composition or feed additive further comprises at least one active ingredient for treating diseases caused by Salmonella infections; the active ingredients for treating diseases caused by Salmonella infections are selected from other types of Salmonella bacteriophage.

19. The pharmaceutical composition or feed additive according to claim 14, wherein the titer of the Salmonella bacteriophage in the pharmaceutical composition or feed additive is ≥10⁵ PFU/mL, preferably ≥10⁸ PFU/mL.

20. An environmental disinfectant comprising the Salmonella bacteriophage, which has the deposit number of CGMCC No. 14332.
